(19) United States
(12) Patent Application Publication
(10) Pub. No.: US 2012/0252116 A1
(43) Pub. Date: Oct. 4, 2012

(54) Fluid Flow Device Containing Nanotubes and Method for Cell Trafficking Using Same

(75) Inventors: Michael R. King, Ithaca, NY (US); Andrew Hughes, Glen Ridge, NJ (US)

(73) Assignee: Cornell University, Ithaca, NY (US)

(21) Appl. No.: 13/500,476

(22) PCT Filed: Oct. 8, 2010
(86) PCT No.: PCT/US10/51914

§ 371 Date: Jun. 18, 2012

Related U.S. Application Data
(60) Provisional application No. 61/249,871, filed on Oct. 8, 2009.

(51) Int. Cl. C12N 5/074; C12N 5/09; C12M 1/00; C07K 14/435; B82Y 5/00

(52) U.S. Cl. 435/325; 435/283.1; 530/395; 977/700

(57) ABSTRACT

The present invention relates to device and method for cell trafficking. In particular, the invention relates to a fluid flow device in which the flow surface has nanotubes immobilized thereon. The nanotubes have molecules on their outer surface, which support cell rolling. Also provided is a method for separation of a cell type from a mixture of different cell types or from a fluid based on the differential rolling property of the cell type on the flow surface.

### CROSS-REFERENCE TO RELATED APPLICATIONS

This application claims priority to U.S. Provisional application No. 61/249,871, filed on Oct. 8, 2009, the disclosure of which is incorporated herein by reference.

### FIELD OF THE INVENTION

The present invention relates to device and methods for cell separation. In particular, the invention relates to separation of selected cell type from a mixture of different cell types or from a fluid based on the rolling ability of the cells on a substrate coated with cell adhesion molecules that support cell rolling.

### BACKGROUND OF THE INVENTION

Purified cell populations have many applications in biomedical research and clinical therapies. Often, cells can be separated from each other through differences in size, density, or charge. However, for cells of similar physical properties, separation is often accomplished by exploiting differences in the presentation of molecules on the cell surface. Cell-affinity chromatography is based on this approach, most often by employing immobilized antibodies to specific cell surface antigens. Such affinity column separations require several distinct steps including incubation of the cells with the antibody, elution of the cells, cell collection, and release of the conjugated antibody, with each step reducing the overall yield of cells and increasing the cost of the process.

There also exists a need for obtaining cellular samples from donors that are enriched in desired biological targets. Because a heterogeneous sample may contain a negligible amount of a biological entity of interest, the limits of separation methods to provide viable and potent biological target in sufficient purity and amount for research, diagnostic or therapeutic use are often exceeded. Because of the low yield after separation and purification, some cell-types, such as stem cells, progenitor cells, and immune cells (particularly T-cells) must be placed in long-term culture systems under conditions that enable cell viability and clinical potency to be maintained and under which cells can propagate (cell expansion). Such conditions are not always known to exist. In order to obtain a sufficient amount of a biological target, a large amount of sample, such as peripheral blood, must be obtained from a donor at one time, or samples must be withdrawn multiple times from a donor and then subjected to one or more lengthy, expensive, and often low-yield separation procedures to obtain a useful preparation of the biological target. Taken together, these problems place significant burdens on donors, separation methods, technicians, clinicians, and patients. These burdens significantly add to the time and costs required to isolate the desired cells. Thus, there continues to be a need for methods and devices for separation/isolation of cells in a continuous, flow-through mode.

### SUMMARY OF THE INVENTION

The present invention provides a device and method for cell trafficking by flowing cells over a flow surface whose topography has been altered by the presence of nanotubes having cell adhesion molecules that support cell rolling attached to their outer surface. By taking advantage of differential rolling velocities of different cell types over such a surface, different populations of cells can be separated.

The device of the present invention has a surface for cell rolling. The surface may be part of a fluidic flow chamber. The surface has nanotubes deposited thereon such that the nanotubes are immobilized on to the surface. The nanotubes in turn have been coated with cell adhesion molecules that support rolling of cells. In one embodiment, the surface has a coating of a composition comprising positively charged molecule (such as polylysine) upon which the nanotubes have been immobilized.

The present invention also provides a method for making a device with altered topography which supports differential rolling of cells such that separation, isolation and/or purification of the cells can be achieved. The method comprises obtaining a fluidic flow chamber having a flow surface. Nanotubes having attached to their outer surface, cell adhesion molecules are then deposited on the flow surface either directly or following coating of the flow surface with a composition comprising positively charged molecules (such as polylysine). In one embodiment, the nanotubes can be deposited on the flow surface and then the nanotubes can be coated with cell adhesion molecules.

The device of the present invention can be used for concentration, separation, isolation and/or purification of cells. The method comprises allowing a fluidic composition comprising cells or mixture of cells to flow along the surface. Because the various cell types roll at different velocities due to the adhesion between particular cells and the coated surface, these cells can be separated, concentrated, or purified from the other cells.

### BRIEF DESCRIPTION OF THE DRAWINGS

FIG. 1. Average rolling velocity of KG1a cells is reduced on halloysite nanotube-coated surface across the physiological range of shear stress. P-selectin was incubated at a concentration of 2.5 μg/mL (A). Average rolling velocity of KG1a cells as a function of the P-selectin incubation concentration at lower (B) or higher (C) shear stress. Errors are SEM (N=3), *** P<0.001, ** P<0.01, * P<0.05.

FIG. 2. The number of cells captured is significantly enhanced as seen in representative micrographs of cells rolling on control (A) and nanotube-coated (B) surfaces. Number of KG1a cells captured per area of surface as a function of the selectin incubation concentration at lower (C) or higher (D) shear stress. Errors are SEM (N=3), *** P<0.001.

FIG. 3. Halloysite nanotube coating on the inner surface of microtubes enhances Colo205 epithelial cancer cell capture as quantified by rolling velocity (A) and the number of cells captured per area of tube surface (B). Errors are SEM (N=3), *** P<0.001.

FIG. 4. Incubation with nanotubes dispersed in media had no effect on the viability of KG1a (A) or Colo205 (B) cells over a 72 h period. “Treated” bars represent the average viability of cells incubated in 10% halloysite nanotube and 90% media, while “untreated” bars represent cells incubated in 10% distilled water and 90% media. Errors are SEM (N=3).

FIG. 5. Schematic of the hypothesized nanoscale surface topography in which individual nanotubes stick up off of the surface and facilitate early cell capture as cells sediment to the surface (A). Representative atomic force microscopy images of halloysite nanotubes immobilized on surfaces after (B) and before (C) treatment to break up and remove large aggregates. This treatment procedure was required to produce more reproducible cell adhesion behavior.

FIG. 6. Comparison of the immunofluorescence of nanotube-coated and control surfaces for a range of P-selectin incubating solution concentrations (A). Representative micrographs of a halloysite-coated tube (B) and control tube (C) both incubated with 10 μg/mL P-selectin solution. Errors are SEM, *** P<0.001.

FIG. 7. The pressure drop across 50 cm nanotube-coated and control tubes was held at a constant value, and the resulting flow rate through either type of tube was calculated. The pressure drop was varied by changing the height of the fluid reservoir relative to the tube outlet, and the results were compared to theory with no adjustable parameters (A). Fluorescent microspheres were perfused through tubes, and the velocity of microspheres near the tube surface was determined as a function of flow rate. Maximum surface roughness heights were determined from AFM data, and the mean maxima were found to be 505 nm on the nanotube coating and 30 nm on the blank control surface. Theoretical microsphere velocities were calculated by eq 1 for surface-to-surface separation distances (6) of 505 and 30 nm, and found to agree with experimental observations with no adjustable parameters (B). Representative surface features from AFM images. The nanotube coating profiles are shifted up 100 nm for ease of viewing (C). Errors are SEM, *** P<0.001.

FIG. 8. Tubes were prepared in an identical manner to the other rolling experiments and then incubated with a blocking anti-P-selectin antibody. A negligible number of cells were adherent in both the control or nanotube-coated tube.

FIG. 9. In another set of experiments, nanotube-coated and control tubes were prepared for rolling experiments and cells were allowed to adhere and roll. EDTA was then introduced to chelate all divalent ions in solution, thereby inactivating the P-selectin protein. After the tubes were gently washed to remove all unbound cells, no cells were observed to remain adhered in the tubes.

FIG. 10. A steep decline in cell capture was observed for P-selectin concentrations below 2.5 μg/mL.

### DETAILED DESCRIPTION OF THE PREFERRED EMBODIMENTS

The present invention provides a device and method for cell trafficking such that populations of cells can be separated based on different velocity of cell rolling. This invention is based on the observation that cell rolling-based trafficking of cells can be enhanced by flowing cells through fluidic chambers in which surface topography is altered with immobilized nanotubes, wherein the nanotubes are functionalized with cell adhesion molecules supporting cell rolling. The presence of nanotubes results in significant changes in the average cell rolling velocity and therefore the number of cells that can be separated out of a fluid flowing through the chamber. It is believed that at least some of the enhanced effect is due to the nanotubes being oriented to extend above the surface and into the flow. This model is supported by data presented herein using atomic force microscopy and immunofluorescence quantification techniques. Thus the nanotubes, while having negligible effect on the macroscopic fluid dynamics, alter the equilibrium streamline of convecting particles or cells. Based on these observations, provided herein are a device and a method for cell trafficking

The device of the present invention comprises a fluidic flow chamber (also referred to herein as a microtube) wherein the inner surface of the chamber has nanotubes immobilized thereon. The nanotubes have cell adhesion molecules supporting cell rolling decorating their outer surface. For cell trafficking, a fluid comprising cells is flowed through the fluidic chamber. Cells expressing ligands complimentary to the cell adhesion molecules on their cell surface roll along the chamber wall at a different velocity than cells not expressing the cell rolling molecules on their cell surface and can therefore be separated out.

Cell rolling or rolling of cells as used herein means when receptor-mediated adhesion causes the translational velocity of the cells to drop below 50% of the freestream hydrodynamic velocity, but the cell translates at least one cell diameter on the surface (i.e., velocity is not zero). For example, the adhesion that initiates leukocyte rolling on the vascular endothelium is referred to as cell rolling. This involves weak affinity interactions between P-selectin and E-selectin (on vascular endothelial cells) and selectin-binding carbohydrate ligands (expressed on circulating hematopoietic stem cells and leukocytes). The cells are considered to be ‘captured’ via the weak interactions and after being ‘captured’, roll slowly over the surface, in contrast to uncaptured cells, which flow faster with the bulk fluid. Thus, cell rolling involves adhesion to the selected cells in a transient manner such that when exposed to the shear rate of a flow field, preferably in the range of 50-1000 s⁻¹ (0.5 to 10 dynes/cm² and all integers and values to the tenth decimal place therebetween) and all integers therebetween, the cells do not bind too tightly to the adhesion molecule, but rather roll along the coated surface. Examples of cell adhesion molecules that support rolling include selectins, cadherins, integrins, and GP-1 or fragments of these molecules, or chimeric of fusion molecules incorporating these molecules that support cell rolling.

In one embodiment, the cell rolling molecule is selectin. Selectins are proteins that hematopoietic stem cells (HSCs) and white blood cells stick to transiently. For example, CD34+ stem cells are the immature stem cells and have maximum stem cell activity, and have been shown to roll more efficiently (or slower) than CD34− stem cells, which are the more committed or differentiated cells. Red blood cells and platelets do not roll on selectin, while white blood cells and some tumor cells exhibit rolling. Examples of selectins are P-selectin, L-selectin and E-selectin as well as recombinant selectin molecules such as P-selectin-IgG chimera and E-selectin-IgG chimera.

Integrins and cadherins are members of family of cell adhesion molecules. Integrins mediate adhesive events important in immune response. These molecules are known to be involved in cell rolling. For example, α4 integrin is considered to mediate leukocyte rolling. Cadherins include E-cadherin, P-cadherin and N-cadherin.

GP1b is a peptide, which is involved in rolling of platelets along the blood vessel walls. The first adhesive interactions between flowing platelets and the vessel wall are considered to be mediated by platelet GP1b transiently binding to the A1 domain of immobilized von Willebrand factor, and is considered to require a threshold level of ˜1 dyn/cm² shear stress to transiently attach, similar to the dynamics of neutrophil adhesion to L- and P-selectin. In general, the lifetime of the GP1b:vWF bond decreases strongly with applied force.

Cell adhesion molecules can be attached or coated on the outer surface of nanotubes by directly physisorbing (absorbing) the molecules on the surface. Another method for attachment of cell adhesion molecules is to first absorb or attach avidin protein (including variants such as “Neutravidin” or “Superavidin”) to the surface, and then reacting this avidin-coated surface with adhesion molecules containing a biotin group. Electrostatic charge or hydrophobic interactions can be used to attach cell adhesion molecules on the surface. Other methods of attaching molecules to surfaces are apparent to those skilled in the art, and depend on the type of surface and cell adhesion molecule involved.

The cell adhesion molecules can be directly attached to the nanotube outer surface. The nanotubes are then immobilized on the surface. By “immobilized” is meant that the nanoparticles will remain attached to the inner surface at shear stress generated by flow of fluid through such as under physiological shear stress (generally between 0.5 to 10 dynes/cm²).

The nanotubes can be made of any material that is generally inert, will remain immobilized to the surface during the flow of the fluid and provide a suitable surface for attachment of the adhesion molecules. In one embodiment, the nanotubes have an average length of from 500 nm to 1.5 μm, are generally hollow and having an average diameter of from 40 to 200 nm. Suitable materials for nanotubes include halloysite, silica and titanium oxide. Nanotubes of halloysite are naturally occurring and therefore easily available. For example, naturally available nanotubes are commercially available from companies such as NaturalNano (Rochester, N.Y.). Nanotubes from halloysite or other materials can also be synthesized.

Naturally occurring nanotubes typically have a length of from 500 nm to 1.2 μm. In one embodiment, at least 50% of the tubes used for coating are from 500 nm to 1.2 μm. In other embodiments, at least 50, 60, 70, 80, 90% or 100% (an all percentages between 50 and 100) of the tubes are from 500 nm to 1.2 μm. The nanotubes typically have a diameter of 40 to 200 nm. In various embodiments, 50, 60, 70, 80, 90 or 100% (and all percentages between 50 and 100) of the tubes have a diameter of from 40 to 200 nm. The nanotubes are generally hollow.

For preparing the fluidic flow chamber, it is preferred that the flow surface be first coated with a composition comprising charged molecules. For example, compositions comprising positively charged molecules such as polylysine or titanium butaoxide can be used. The nanotubes are then deposited on to the flow surface. To avoid the nanotubes being deposited as clumps, the compositions comprising nanotubes can be filtered or clumps removed by other suitable means. For example, the solution comprising halloysite tubes can be sonicated (such as for 1 minute) and then filtered (such as with a 0.45 μm filter). If the nanotubes are allowed to form multilayer coatings, the cell behavior and capture becomes unpredictable. Thus, monolayers are preferred with some nanotubes projecting into the lumen. When the nanotube concentration was diluted prior to coating, cell capture was observed to decrease indicating a monolayer formation. Desired cell adhesion molecules (such as selectins) are attached to the outer surface of the nanotubes. The attachment of the selectins can be carried out either before or after removal of the clumps, although in a preferred embodiment the selectin attachment is carried out after the clumps are removed.

The nanotubes may or may not cover the entire flow surface. Some of the nanotubes extend above the flow surface. In one embodiment, the nanotubes extend up to the length of a nanotube above the flow surface. In another embodiment, the nanotubes extend at least by 50 nm. Thus, nanotubes can extend from 50 nm to 1.2 μm into the lumen of the fluidic chamber. In various embodiments, the nanotubes extend up from 50 nm to less than 1.1 μm, 1.0 μm, 900 nm, 800 nm, 700 nm, 600 nm and all integers therebetween into the lumen above the flow surface. In one embodiment, the nanotubes extend between 40 nm to 1.2 μm and all integers and ranges therebetween.

In one embodiment, the invention exploits the rolling properties of cells to separate them from other cell types or from fluid components. Thus, cell types which exhibit rolling behavior can be separated from those that do not exhibit rolling but rather flow along the fluid stream. Additionally, cell types can also be separated based on differential rolling velocities, which may be the result of different numbers or types of cell adhesion molecule ligands on cells.

In one embodiment, the invention exploits the natural rolling properties of hematopoetic stem cells (HSCs), separating them from other blood cells. In this embodiment, the blood cells are rolled along a surface coated with nanotubes, in turn decorated with selectin proteins. The adhesion between the selectins and the HSC retards the rolling rate of HSC along the surface, while other cells roll or flow at their normal rate. The difference in rolling rates concentrates and separates the HSCs from the other cells.

Separation of HSCs can be useful in the treatment of many cancers, hematological, and immunodeficiency diseases. The treatment of cancers and immune diseases generally requires aggressive radiation and chemotherapy that kills healthy bone marrow required for blood production. Bone marrow and peripheral HSC transplantation enables doctors to replace the diseased or destroyed bone marrow with health marrow that produces normal blood cells. The present device will allow separation of HSC's out of the peripheral blood supply for later readmission to the body

The present invention can also be used to capture circulating tumor cells (CTCs)—cells which have detached from a primary tumor and circulate in the bloodstream. Some of these CTCs have the potential to eventually form additional tumors at different sites and different tissue(s). Moreover, these cells could also be released as a tumor is excised and some CTCs are set free during the surgery. Thus, the device and method of the present invention can be used to capture the CTCs. Additionally, the invention can also be used to clean the blood, as a diagnostic tool to see if CTCs are present or even capture these CTCs and re-engineer/re-introduce back into the host to use/fight against the tumor.

In an embodiment of the present invention, the device of the present can be used as an implantable device for in vivo cell separation, concentration, and/or purification of cells in a bodily fluid. The implantable device refers to any article that may be used within the context of the methods of the invention for changing the concentration of a cell of interest in vivo. An implantable device may be a stent, catheter, cannula, capsule, patch, wire, infusion sleeve, fiber, shunt, graft, and the like. An implantable device and each component part thereof may be of any bio-compatible material composition, geometric form or construction as long as it is capable of being used according to the methods of the invention.

The device may contain a recycle stream where part of the outlet stream from the device is recycled back to the inlet stream. This effectively increases the inlet concentration of the desired cells, thus improving the concentration of the outlet stream.

In another embodiment, the device may contain multiple stages of flow chambers in series. In this case, at least two devices are connected in series, where the outlet stream of one device feeds into and inlet of the next device. Each subsequent device further concentrates, separates, and/or purifies the desired cells.

In one embodiment, the flow chambers are constructed such that, instead of producing a well-defined parabolic velocity profile, the flow represents the complex sinusoid flow in the bone marrow.

In one embodiment, delivery molecules can be provided in the lumen of the nanotubes for delivery to cells during rolling. Because the inner and outer faces of halloysite nanotubes carry a net negative charge while the edges are amphoteric, the nanotubes can be used for encapsulation and sustained release of drugs, particularly, cationic drugs. Thus, a cationic drug would be loaded into the nanotubes, and the nanotubes would then be coated onto the inner surface of microtubes followed by a functionalization step in which a cell adhesion molecule (such as a selectin) is attached to the outer surface of the nanotubes. In one embodiment, the functionalization of nanotubes can be done before attaching the nanotubes to the inner surface of the microtubes. Targeted cells that are able to roll on the nanotube coating will experience a microenvironment with a relatively high concentration of drug that is steadily being released from the nanotubes. In an alternative approach a therapeutic drug could be loaded into nanotubes, which could then be loosely bound to the flow surface. Targeted cells rolling on the flow surface would bind to the nanotubes and ingest the nanotubes, thus internalizing the loaded therapeutic. Cationic drugs are the best candidates due to the net charge on nanotubes, and suitable therapeutics include doxorubicin hydrochloride, irinotecan hydrochloride, and cationic antimicrobial peptides (CAPs), and the like.

The following example is given to illustrate the present invention. It should be understood that the invention is not to be limited to the specific conditions or details described in this example.

### EXAMPLE 1

### Materials and Methods

### Reagents and Antibodies

RPMI 1640 cell culture media, fetal bovine serum, penicillin-streptomycin, phosphate buffered saline (PBS), Hank's balanced salt solution (HBSS), and 1× trypsin were purchased from Invitrogen (Grand Island, N.Y.). Recombinant P-selectin-IgG chimera and recombinant E-selectin-IgG chimera were obtained from R&D Systems (Minneapolis, Minn.). Halloysite nanotubes in water (6.6% by weight) were provided by NaturalNano (Rochester, N.Y.). Trypan blue stain (0.4%) was obtained from Lonza (Wilkersville, Md.). Poly-L-lysine (0.1% w/v in water) was obtained from Sigma-Aldrich (St. Louis, Mo.). Blotting grade blocker nonfat dry milk was obtained from Bio-Rad Laboratories (Hercules, Calif.). Mouse anti-human CD62P (P-selectin) monoclonal IgG was obtained from eBioscience (San Diego, Calif.). Alexa Fluor 546 donkey anti-mouse IgG (H+L) antibody was obtained from Invitrogen (Carlsbad, Calif.).

### Cell Lines and Cell Culture

Acute myeloid leukemic KG1a cell line (ATCC number CCL-264.1) and colon cancer Colo205 cell line (ATCC number CCL-222) were obtained from ATCC (Manassas, Va.). These cell lines were cultured in RPMI 1640 media supplemented with 2 mM 1-glutamine, 25 mM HEPES, 10% (v/v) fetal bovine serum, and 100 U/mL penicillin-streptomycin (complete media) at 37° C. and 5% CO₂ under humidified conditions.

### Preparation of Cells for Rolling Experiments

Colo205 cells, an adherent cell line, were trypsinized for 5 min and then allowed to incubate for up to 5 h before use to ensure normal surface receptor expression. Both KG1a and Colo205 cells were washed twice with 1× PBS at 1100 rpm in an Allegra X-22 refrigerated centrifuge at 4° C. and resuspended in the flow buffer at a concentration of 10⁶ cells/mL. The flow buffer was composed of PBS containing Mg²⁺ and saturated with Ca²⁺. At least 90% viability of cells was confirmed by trypan blue stain.

### Preparation of Halloysite Nanotube Solution

Stock halloysite solution was treated to break up and remove large aggregates. Stock solution was vigorously mixed and subjected to a sonic dismembrator obtained from Fisher Scientific (Pittsburgh, Pa.). The resulting solution was then filtered through a 0.45 μm pore size PVDF membrane syringe filter (Pall Life Sciences, Port Washington, N.Y.).

### Preparation of Surfaces

Recombinant human P- or E-selectin-IgG chimeric protein was dissolved in PBS to 20 μg/mL. The surface was first washed with 75% ethanol and then distilled water. Control surfaces were incubated for 2 h with P- or E-selectin-IgG diluted to concentrations of 2.5-10 μg/mL and then incubated with 5% milk protein in PBS for 1 h. Finally, the immobilized selectin molecules were activated by incubation with calcium-containing flow buffer for 10 min. Nanotube-coated surfaces were incubated with 2:8 poly-L-lysine solution (0.02% w/v) for 5 min, and then the treated nanotube solution was incubated for 3 min. The nanotube-coated surfaces were then coated with P- or E-selectin-IgG and milk protein in the same manner as the control surfaces. All incubations were carried out at room temperature.

### Rolling Experiments

Micro-Renathane microtubing (300 μm i.d.) was obtained from Braintree Scientific (Braintree, Mass.), cut to a length of 50 cm, and secured to the stage of the Olympus IX81 motorized inverted research microscope (Olympus America, Melville, N.Y.) after surface functionalization as described above. A CCD camera (Hitachi, Tokyo, Japan) and DVD recorder (Sony Electronics) were used to record experiments for offline analysis. Flow of cell suspension containing cells at a concentration of 10⁶ cells/mL in flow buffer through the microtubes was controlled via a syringe pump (KDS 230, IITC Life Science, Woodland Hills, Calif.). Cells were loaded in the microtubes at a shear stress of 2.5 dyn/cm² for 5 min prior to performing flow experiments. Shear stress values of 2.5-6.67 dyn/cm² were then initiated and flow was allowed to establish for 1 min prior to data collection.

### Viability Assays

Viability assays were performed on KG1a and Colo205 cells in triplicate in which treated cells were incubated for 72 h in media containing 10% treated halloysite nanotube solution. Viability counts were performed at the beginning and end of the 72 h period on a hemacytometer (Hausser Scientific, Horsham, Pa.) using trypan blue stain. Cells were initially diluted to a concentration of 2.5×10⁵ cells/mL.

### Atomic Force Microscopy

Flat samples of halloysite nanotube-coated surfaces were prepared for atomic force microscopy by coating glass coverslips following the same method used to coat the microtubes. Surfaces were prepared using nanotube solutions before and after being treated by the methods described above. New tubes were sectioned into planar substrates for imaging of the inner surface. Samples were then imaged using a Veeco DI-3000 atomic force microscope. Images of 10 μm×10 μm were recorded at five random locations on each sample and surface topography, as well as phase shift data were recorded and analyzed off line using Image SXM 189 software for Mac OS. Three images each of the flat nanotube-coated samples and untreated tube samples were analyzed in Image SXM to inspect the surface height profiles. This was done across the entire image at 20 random positions per image.

### Antibody Blocking Experiments

Nanotube-coated and control surfaces were prepared as described above. P-selectin-IgG was diluted to 2.5 μg/mL in PBS− and incubated inside the microtube for 2 h at room temperature (RT). The microtube was then blocked with 5% milk protein solution in PBS− for 1 h at RT. The P-selectin in the microtube was activated by incubation with PBS+ saturated with Ca²⁺ for 15 min. Mouse anti-human CD62P (P-selectin) AK-4 monoclonal antibody was diluted to 100 μg/mL in PBS+ and incubated inside the microtube for 2 h at RT. Cell suspension containing KG1a cells at a concentration of 10⁶ cells/mL in flow buffer was then perfused through the microtube at low shear (2.5 dyn/cm²), and cellular behavior under flow was observed by video microscopy.

### Ca²⁺ Chelation Experiments

Nanotube-coated and control surfaces were prepared in the same manner as those prepared for rolling experiments, with an incubating concentration of P-selectin-IgG protein of 2.5 μg/mL in PBS− (2 h) and blocked with 5% milk protein (1 h). After activation of P-selectin with PBS+ saturated with Ca²⁺, KG1a cell suspension at a concentration of 10⁶ cells/mL was perfused through the tubes at a shear stress of 2.5 dyn/cm² for 5 min. Flow was then stopped, and the syringes in the syringe pump used to withdraw the cell suspension from the cell source through the tubes were replaced with syringes containing 5 mM EDTA (VWR Inc., West Chester, Pa.) in PBS−. The syringe pump was switched from withdraw to infuse and a tube volume was pumped slowly through each tube. The EDTA solution was allowed to sit in the tubes for 10 min, and then another tube volume of EDTA solution was pumped slowly through the tubes to clear unbound cells. The tubes were then scanned using video microscopy for the presence of adherent cells.

### P-Selectin Surface Density Measurements

Eight tubes were cut to a length of 20 cm. Four tubes were coated with halloysite nanotubes, and four were left uncoated as control tubes. Three nanotube-coated tubes and three control tubes were coated with varying concentrations of P-selectin-IgG in PBS− for 2 h and then blocked for 1 h with 5% milk protein. The remaining nanotube-coated tube and control tube were incubated with PBS− for 2 h and then blocked with 5% milk protein for 1 h. All tubes were then incubated with PBS+ saturated with Ca²⁺ to activate the adsorbed P-selectin and then with 100 μg/mL mouse anti-human CD62P (P-selectin) IgG in PBS+ for 2 h. The tubes were then washed thoroughly with PBS+, and then a solution of 200 μg/mL donkey anti-mouse IgG in PBS+ was incubated in all of the tubes for 2 h protected from light. The tubes were then washed thoroughly with PBS+. One tube at a time was placed on the microscope stage to avoid unequal photobleaching, and prior to being placed on the microscope either end of the tube was sealed using surgical clamps. Fluorescence micrographs were taken using a 4× objective so that a large area of background could be observed along with a large area of tube. Fifteen micrographs were collected at random locations along the length of each tube per tube. The exposure time for each image was set to 300 ms. Micrographs were analyzed off-line in ImageJ, with the regions of interest outlined and histograms of the brightness intensity within the regions of interest quantified. Mean intensity was determined from the histograms along with standard deviation. Individual micrographs were analyzed by subtracting the intensity of the regions outside the tube. Relative fluorescence intensity values were then corrected by the mean brightness values observed in the tubes that were not coated with P-selectin.

### Pressure Drop Experiments

A 50 cm tube was coated with halloysite nanotubes as described above and compared with 50 cm uncoated control tubes. A 75 mL reservoir was connected to a tube and initially suspended using a ring stand so that the tube outlet reached the benchtop. The vertical distance between the tube outlet at the benchtop and the 75 mL mark in the reservoir was initially set at 84 cm. The reservoir was then filled to the 75 mL mark with water, and this water level was manually maintained throughout the experiment. The tube outlet was placed in a dry weigh boat as a stop watch was simultaneously started and flow effluent was collected for 5 min, after which the tube outlet was immediately removed from the weight boat and the weigh boat was weighed to determine the volume of water that flowed through the tube. This was repeated three times for each tube at each of four heights: 84, 74, 64, and 49 cm.

### Microsphere Perfusion Experiments

Nanotube-coated and control tubes were prepared as described above, coated with 2.5 μg/mL for 2 h and blocked for 1 h. Fluorescent microspheres with mean diameter of 1.9 μm and an emission wavelength of 520 nm (Bangs Inc., Fishers, Ind.) were suspended in flow buffer at a concentration of 5×10⁵ microspheres/mL and perfused through the tubes at various flow rates. For each flow rate, a location along either tube was chosen at random and the surface was brought into focus using a 20× objective with a 1.6× magnification changer engaged. Epifluorescence mode was then used to take 100 time lapse micrographs for times ranging from 10 to 75 ms, with 500 ms intervals between each micrograph, using a TRITC filter set. This was repeated so that 100 micrographs were recorded at three random locations along the length of each tube for each of the four flow rates examined: 0.03, 0.06, 0.095, and 0.13 mL/min. Microsphere velocity was determined by measuring the length of the in-focus streaks made by translating microspheres that were close to the tube surface. Measurements were taken using ImageJ, and the scale was determined using a slide micrometer (Olympus, Tokyo, Japan).

### Data Analysis

Rolling velocity was calculated by measuring the distance a rolling cell traveled over a 30 s interval. Rolling cells were defined as cells traveling in the direction of flow at an average velocity less than 50% of the hydrodynamic free stream velocity. Videos of rolling cells were taken at three random locations along the microtube. The quantity of cells adherent to the surface was determined by recording micrographs at 30 random locations along the microtube. All errors are reported as standard error of the mean, and statistical significance was determined by unpaired t test using GraphPad Prism (GraphPad Software, San Diego, Calif.).

### Results

### Halloysite Nanotube Coating Reduces Cancer Cell Rolling Velocity

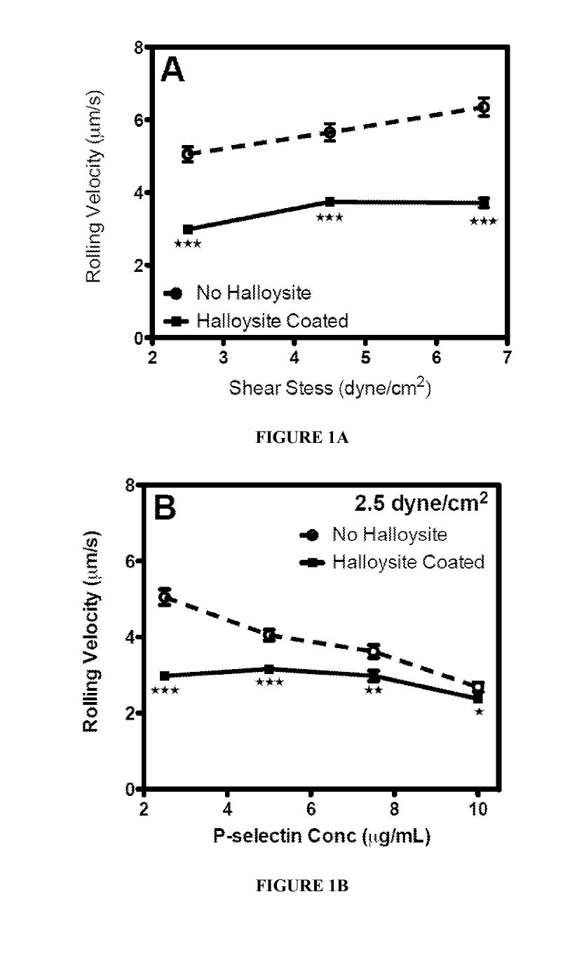

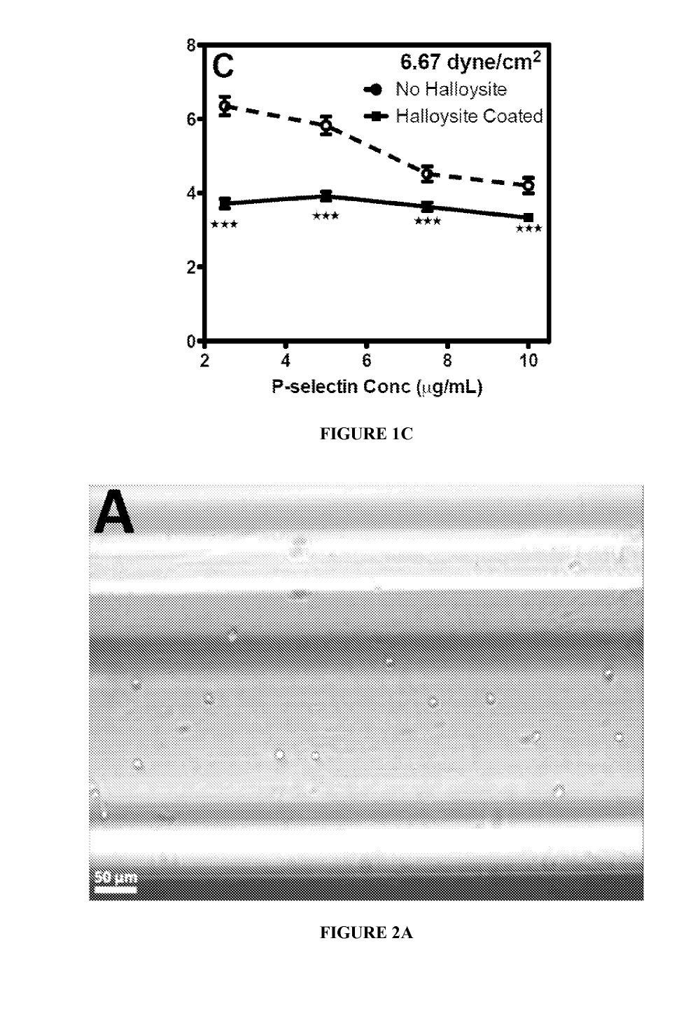

Cell suspensions containing KG1a cells in flow buffer were perfused through capillary tubes at a range of flow rates imparting known shear stresses on the inner surface of the tubes. Tubes coated with halloysite nanotubes coated with P-selectin were compared to tubes coated with P-selectin alone, for a P-selectin incubating solution concentration of 2.5 μg/mL. The average rolling velocity of KG1a cells in the nanotube-coated tubes was significantly reduced, when compared to control tubes, across the range of shear stresses (FIG. 1A).

Reduction of Rolling Velocity Caused by Nanotube Coating Attenuates with Increased P-Selectin Surface Density

The average rolling velocity of KG1a cells on the nanotube-coated surfaces was compared to that on control surfaces for a range of P-selectin surface densities. It was determined that the average velocity of rolling cells on the nanotube-coated surfaces was significantly lower than that on the control surfaces; however, the degree to which the average rolling velocity is reduced decreases as the surface density of P-selectin is increased. This was seen at both lower and higher shear stress (FIGS. 1B and C, respectively). It was also determined that increasing the surface density of P-selectin significantly affected rolling velocity on the control surface, but had little effect on rolling velocity on the halloysite-coated surface at both lower and higher shear stress.

### Halloysite Nanotube Coating Increases the Number of Captured Cells

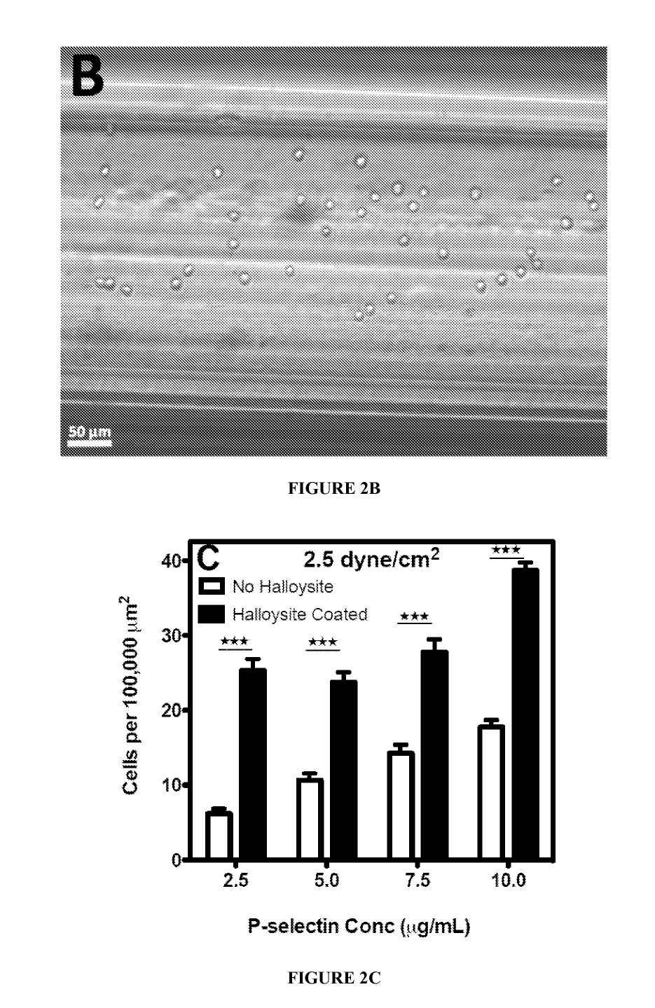

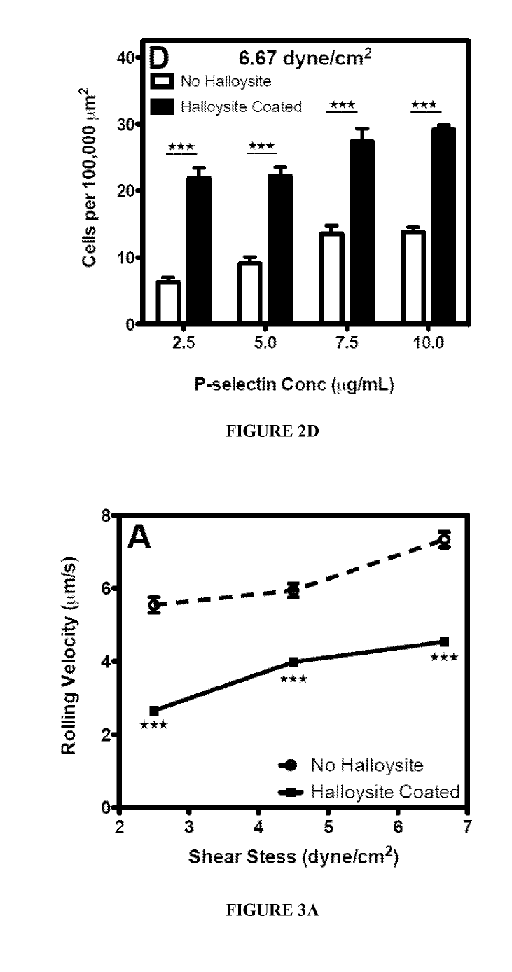

The number of cells both rolling and statically adhered to the tube surface is a useful indication of the effectiveness of the surface at capturing target cell populations. The number of cells adhered to the inner surface of the tubes was analyzed as a function of shear stress as well as P-selectin surface density. A significant increase in the number of cells captured on the nanotube-coated surface was discovered (FIG. 2A,B) for all P-selectin surface densities at both lower and higher shear stress (FIGS. 2C and D, respectively). Interestingly, the effect of the nanotube coating was found to be insensitive to the surface density of P-selectin.

### Epithelial CTC Exhibit Similar Behaviors on Nanotube-Coated Surfaces

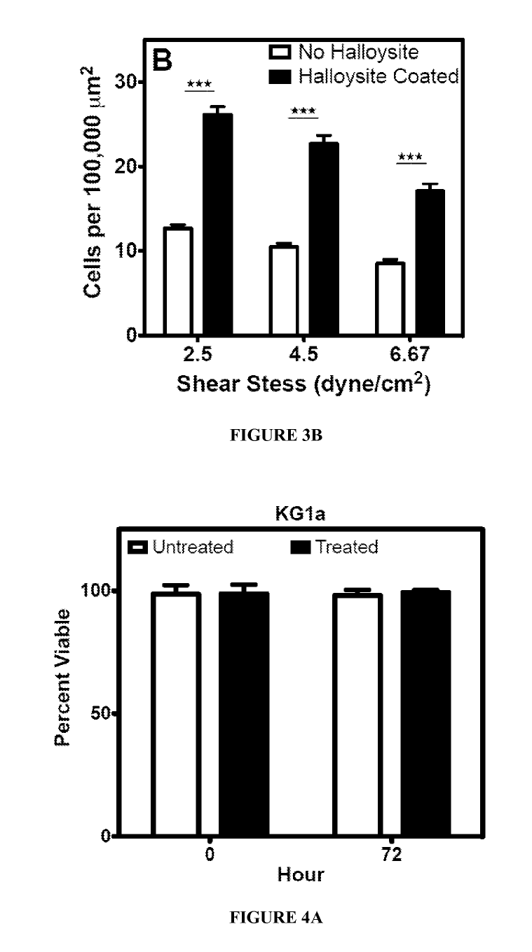

Colo205 colon carcinoma cells were perfused through tubes coated with halloysite nanotubes and E-selectin as well as tubes coated with E-selectin alone, and their behavior was compared over a range of shear stresses. Colo205 cells were used as a model of epithelial cancer CTC. For these experiments, the concentration of the E-selectin incubating solution was held constant at 2.5 μg/mL. The reduction in both the average rolling velocity as well as the increase in the number of adherent cells due to the halloysite nanotube coating for Colo205 cells was found to be similar to those of KG1a cells (FIGS. 3A and B, respectively).

### Halloysite Nanotubes Do Not Affect Cell Viability

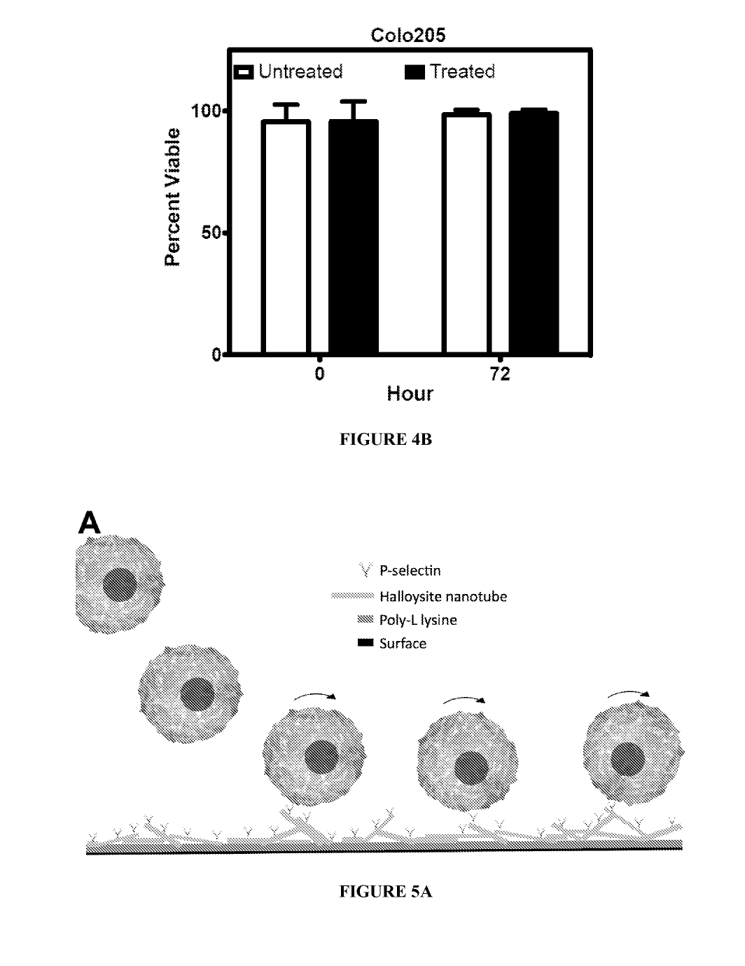

Cells were cultured with and without halloysite nanotubes dispersed in their media, and cell viability was measured after 72 h of incubation at 37° C. and 5% CO₂ at humidified conditions. Treated cells were those cultured in 10% nanotube solution and 90% media, while untreated cells were cultured in 10% distilled water and 90% media. As shown in FIGS. 4A and 4B, after 72 h incubation, neither KG1a nor Colo205 cells were affected by the presence of nanotubes in the media.

AFM Shows Nanotubes Extend above the Surface

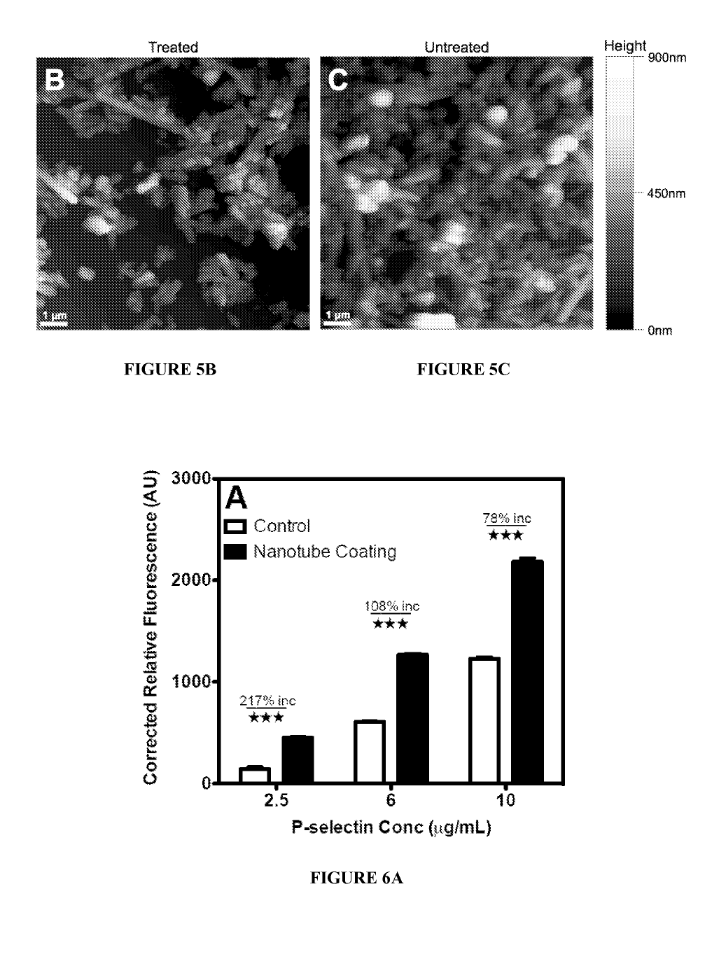

Atomic force microscopy images taken of nanotubes coated on a thin layer of poly-L-lysine show that nanotubes are oriented in such a way that they extend above the surface a distance of hundreds of nanometers up to a micrometer. AFM images were taken of untreated nanotubes (FIG. 5B) as well as treated nanotubes (FIG. 5C), and it was found that the treatment procedure was effective at breaking up and removing large aggregates and thus is substantially free of aggregates; however, the height to which the nanotubes extended above the surface was largely preserved.

### Immunofluorescence Labeling Shows Increased P-Selectin Adsorption on Nanotube Coating

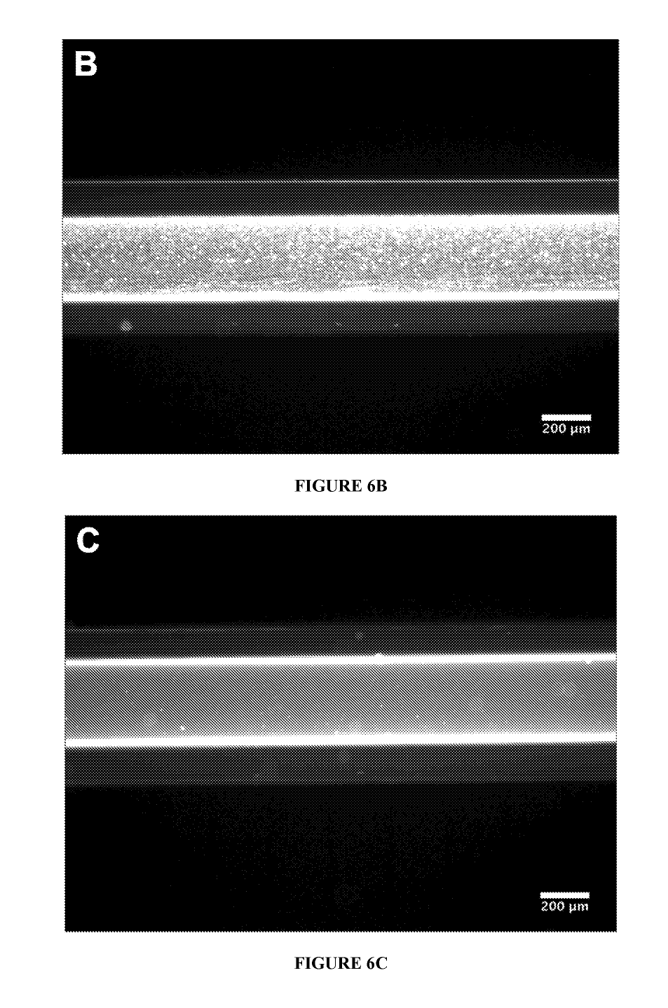

Fluorescence microscopy of tagged antibodies specific to P-selectin shows that the surface density of P-selectin adsorbed onto the nanotube-coated surfaces is significantly greater than that on control tubes (FIG. 6A). The relative difference in P-selectin surface density due to the nanotube coating attenuates as the concentration of the incubating P-selectin solution is increased. Representative micrographs are shown in FIGS. 6B and C. The fluorescence intensity value from each image was calculated relative to background brightness, and mean fluorescence values for the tubes were subsequently corrected by the small amount of fluorescence seen due to tube autofluorescence or nonspecific antibody binding.

### Specificity of Selectin-Mediated Cell Capture

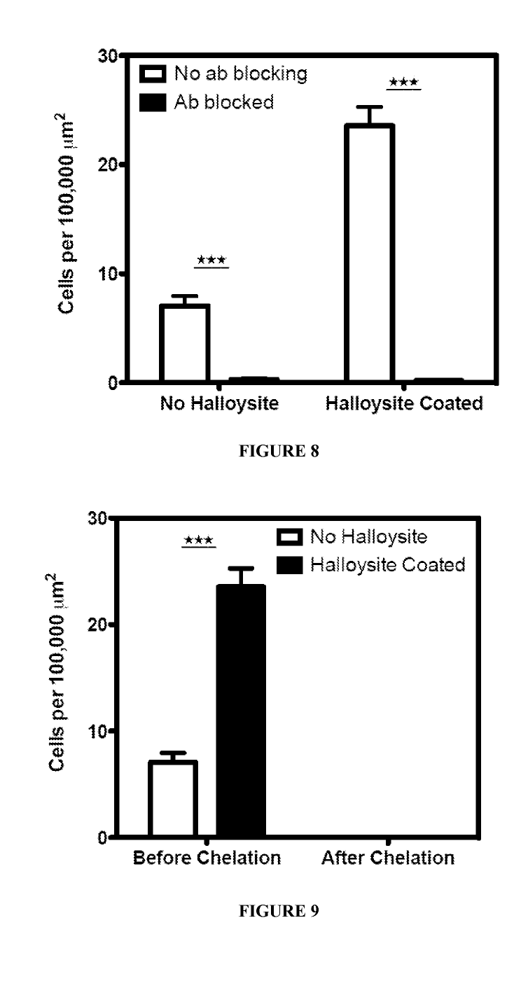

In one set of experiments, tubes were prepared in an identical manner to the other rolling experiments and then incubated with a blocking anti-P-selectin antibody. A negligible number of cells were adherent in both the control or nanotube-coated tube (FIG. 8). In another set of experiments, nanotube-coated and control tubes were prepared for rolling experiments and cells were allowed to adhere and roll. EDTA was then introduced to chelate all divalent ions in solution, thereby inactivating the P-selectin protein. After the tubes were gently washed to remove all unbound cells, no cells were observed to remain adhered in the tubes (FIG. 9).

### Halloysite Nanotube Coating Does Not Alter the Macroscale Fluid Dynamics

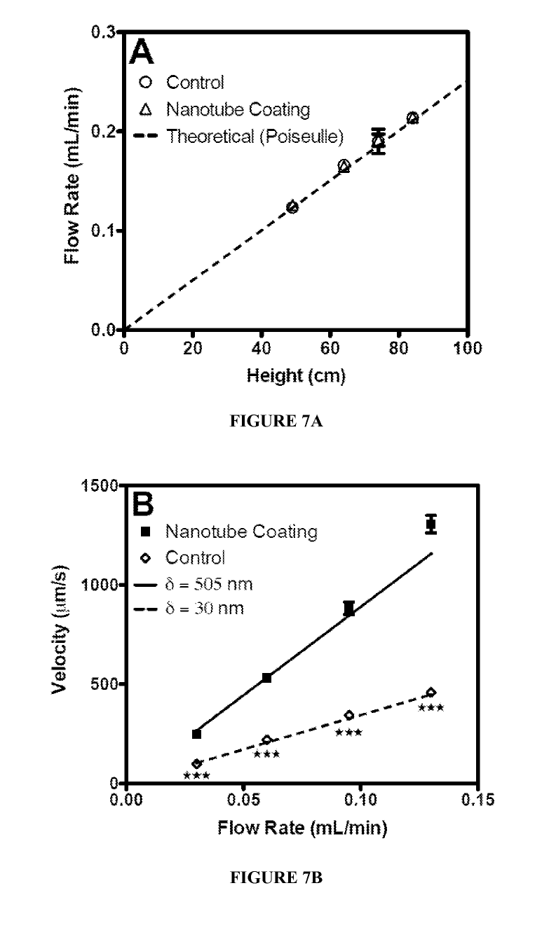

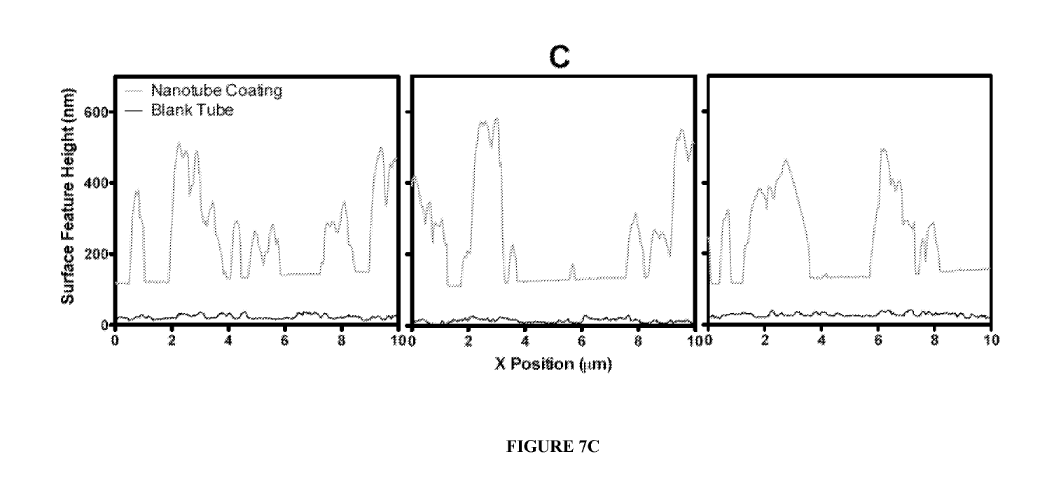

Tubes of 50 cm in the presence or absence of a nanotube coating were subjected to a constant hydrostatic pressure drop. At four different reservoir heights, the flow rate through each tube was determined by weighing the fluid collected at the tube outlet over a 5 min period. The flow rates in the nanotube-coated and control tubes were calculated, and the mean flow rates were found to differ by only 0.18% at a reservoir position of 84 cm, 0.71% at 74 cm, 0.77% at 64 cm, and 2.1% at 49 cm. Theoretical flow rates were calculated using the Hagen-Poiseuille equation, and the experimental values were shown to agree very well with theory (FIG. 7A).

### Halloysite Nanotube Coating Alters Surface Separation Distance of Flowing Particles

Fluorescent microspheres were perfused through nanotube-coated and control tubes at varying flow rates in order to obtain a local measurement of fluid velocity and wall shear rate. Time lapse fluorescence microscopy enabled calculation of individual microsphere velocities. The mean microsphere velocity was found to be significantly higher in the nanotube-coated tube than in the control tube, and the rate of increase of microsphere velocity seen with the increasing perfusion rate was found to be greater in the nanotube-coated tube (FIG. 7B).

Several AFM images of nanotube-coated surfaces and untreated tube surfaces were analyzed to characterize their nanoscale topography (FIG. 7C). The maximum surface feature height was evaluated in 20 random slices of the AFM images, and the mean maximum surface feature in the control tube was found to be ˜30 nm, while the mean maximum feature height on the nanotube-coated surface was found to be ˜505 nm. Since microspheres cannot flow any closer to the tube surface than the tallest roughness elements on the surface, the mean maximum feature height can be employed as limiting surface-to-surface separation parameters. The theoretical velocity of a microsphere translating at a specified separation distance from a plane wall can be calculated based on the Stokes' flow solution for a sphere near a wall in shear flow

$$\frac{U}{hS} \sim \frac{0.7431}{0.6376 - 0.200 ln ( δ / a )} \qquad ( 1 )$$

where U is the microsphere velocity, h is the distance between the center of the microsphere and the wall, S is the shear rate, δ is the distance between the microsphere surface and the wall (the separation distance), and α is the microsphere radius (FIG. 7B). Prediction of the microsphere velocity based on the measured surface roughness agreed well with experimental observations with no adjustable parameters. This suggests that the microspheres translating over the nanotube-coated surface are translating in the same velocity field as those flowing over the control surface; however, they are on a different streamline as forced by the larger roughness elements.

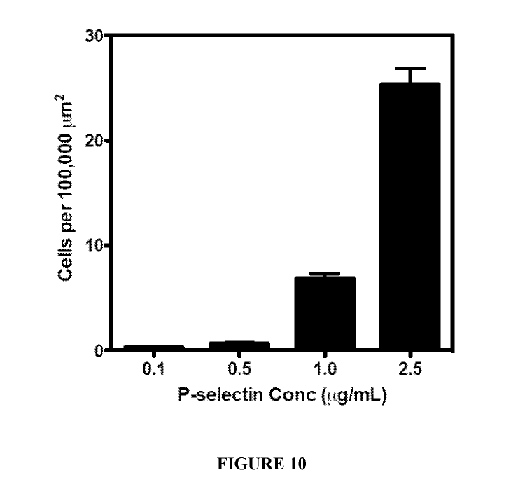

In this example, we have demonstrated that halloysite nanotube coatings can significantly enhance selectin-mediated cell adhesion to a microtube surface under flow, and that the cellular adhesion is mediated specifically by selectin interaction (FIGS. 8 and 9). Rolling velocity was found to increase and the number of cells captured was found to decrease with increased shear stress. This is possibly because increased shear stress imparts more force acting against the bonds between selectin molecules and its cell surface ligand as a cell rolls along a surface. Further, rolling velocity profiles were found to have a close correlation to the nanotube coating concentration, shifting to faster velocities as the nanotube coating was increasingly diluted (data not shown). Additionally, a steep decline in cell capture was observed for P-selectin concentrations below 2.5 μg/mL (FIG. 10).

We found that, at low surface densities of selectin protein, there was a large difference in the rolling velocity between nanotube-coated and control surfaces, and this difference in rolling velocity decreased as the selectin surface density increased (FIGS. 1B and C). This phenomenon could be explained by a saturation effect on the nanotube coating. When the relatively large sized halloysite nanotubes adhere to a surface the total area of the surface is necessarily increased, providing more area onto which selectin molecules can absorb. Thus, for a given incubation concentration of selectin, there would be a greater macroscopic surface density of selectin protein on the nanotube-coated surface. An increased surface density of selectin protein would then result in a decreased rolling velocity due to a greater average number of bonds per cell and more bonds that must break for the cell to continue rolling.

Immunofluorescent measurements support the hypothesis that P-selectin density is significantly higher on the nanoparticle-coated surfaces, and that this difference is attenuated at the highest P-selectin incubation concentrations (FIG. 6A). It is important to note that the P-selectin antibody used is specific to the carbohydrate-recognition domain (CRD) of P-selectin, the domain of P-selectin which binds to cells. Therefore, the assay detects only those P-selectin molecules that are available for binding in the proper orientation.

The number of adherent cells on the surfaces was additionally investigated to characterize the impact of the nanotube coating. Significant enhancement in capture was observed for all conditions. However, as the selectin surface density on the surfaces was increased, the effect of the nanotube coating was not found to attenuate as it did with rolling velocity. Consequently, the straightforward explanation of increased surface area caused by the nanotubes does not fully explain this trend because there is no saturation of the number of cells captured on the surfaces.

A likely explanation for the observed phenomena takes into account the reported dimensions of the nanotubes: nanotubes are situated such that they stick up off of the surface, presenting selectin molecules farther out into the flow profile (FIG. 5A). Due to hydrodynamic lubrication forces, the cell sedimentation time scale increases as 1/δ (where δ is the surface-to-surface separation distance) as it approaches the wall. Thus, it is possible selectin molecules are presented into this lubrication region close to the surface, and flowing cells that would otherwise require more sedimentation time to contact the surface are captured earlier and brought to the surface and proceed to roll. Therefore, as the selectin incubation concentration is increased, more selectin molecules are presented into the flow field and cells are captured at a higher rate. This phenomenon would not be produced by adding more selectin to a flat surface, and thus, the effect of halloysite on the number of cells captured does not diminish.

Atomic force microscopy was performed to investigate the orientation of nanotubes on the surfaces and it was found that nanotubes indeed extend above the surface by several hundred nanometers (FIG. 5B). It was also found that the treatment procedure developed for the stock halloysite solution that was required to produce homologous solutions for all experiments did not significantly change the topography of the nanotube coating, as those nanotubes were raised to a similar height above the surface with a comparable surface density of peaks (FIG. 5C).

The effect that the nanotube coating has on the fluid dynamics within the microtubes was examined in two separate experiments designed to probe both the macroscopic and microscopic flow behavior. In one experiment, the flow rate was measured while pressure drop across the tube length was set to a constant value by maintaining the fluid level in a reservoir. The reservoir then moved to different heights to create different constant pressure drops. Negligible difference in the bulk flow rate was observed in the nanotube-coated and control tubes. A range of Reynolds numbers from 2 to 15 was studied, which extends beyond the range of flow rates used in the adhesion experiments. This is well within the laminar regime, and thus, the friction factor is expected to be independent of surface roughness. It follows, then, that the Hagan-Poiseuille equation can be used to estimate the fluid flow rate in either tube, and comparison with experimental results confirms this (FIG. 7A). The Hagan-Poiseuille equation for laminar flow of a viscous, incompressible fluid through a tube relates the pressure drop and volumetric flow rate as

$$Δ P = \frac{8 μ L Q}{π {r}^{4}} \qquad ( 2 )$$

where ΔP is pressure drop, μ is the dynamic viscosity of the fluid, L is the tube length, Q is the volumetric flow rate, and r is the tube radius. Since ΔP, μ, and L are controlled in the experiment and Q was found to be identical between tubes, we may conclude that the tubes have an equal hydraulic radius.

The microscale fluid dynamics was examined in nanotube-coated tubes and compared to those in control tubes. Time lapse video microscopy of fluorescent microspheres initially suggested that the fluid dynamics close to the tube surface is different, due to the observation that microspheres travel faster in nanotube-coated tubes than in control tubes. However, since it was previously determined that the bulk fluid flow corresponds to the same tube diameter with or without the nanotube coating, another explanation for this observation is that the microspheres in the nanotube-coated tube are translating on a streamline that is farther away from the tube surface.

A negatively buoyant particle flowing along a surface can only approach as close to the surface as the largest roughness features on the surface. This is evident when the sedimentation velocity of a microsphere close to a surface is considered. The sedimentation velocity can be calculated using Brenner's correction for Stokes' law as implemented by Smart and Leighton (Phys. Fluids A, 1989, 1(1): 52-60.)

F=6πμα²U_{S}λ (3)

where U_{S} is the sphere sedimentation rate and is the correction term

$$\begin{matrix}λ = \frac{4}{3} sinh ( α ) \times {\sum }_{n = 1}^{∞} [ ( \frac{n ( n + 1 )}{( 2 n - 1 ) ( 2 n + 3 )} ) ( \frac{2 sinh ( 2 n + 1 ) α + ( 2 n + 1 ) sinh ( 2 α )}{4 {sinh}^{2} ( n + \frac{1}{2} ) α - {( 2 n - 1 )}^{2} {sinh}^{2} ( α )} - 1 ) ] & ( 4 ) \\ α = {cosh}^{- 1} ( 1 + δ ) = ln ( 1 + δ + \sqrt{δ ( 2 + δ )} ) & ( 5 )\end{matrix}$$

By performing a force balance on the microsphere, balancing the corrected drag force and the net buoyancy force, 4/3πα³Δρg, the sedimentation velocity is predicted to be 5×10⁻⁵ nm/s at δ=505 nm and 3×10⁻⁶ nm/s at δ=30 nm. Considering that the microspheres are translating on the order of 10²-10³ μm/s, and encountering roughness features on the order of one every 10 μm, the microspheres are expected to translate at a constant distance from the surface, with the distance defined by the tallest roughness features. The height of nanotubes sticking into the flow is sufficient to explain the separation distance of microspheres flowing over them, providing further evidence that the fluid flow field in the tube is unaltered by the presence of the nanotube coating, whereas the particle/cell convection will be altered (FIG. 7C). Therefore, the shear rate in the tube, and the shear stress at the tube surface as predicted by Poiseuille Law, for a given flow rate is equivalent to smooth surfaces. Since the tube radius is about 150-fold larger than that of a microsphere, and about 150-fold larger than the characteristic 6, the error in assuming a planar geometry is negligible.

In contrast to some earlier reports of nanoparticles being cytotoxic to cells, the halloysite nanoparticles were found to be non-toxic (FIG. 4). This finding, coupled with the equally enhanced capture of leukemic and epithelial CTC, indicates that halloysite nanotube coatings provide an effective and practical method for enhancing cancer cell capture and ultimately promises to advance the feasibility of individualized cancer treatment

While the present invention has been described through specific examples, routine modifications to the various embodiments will be apparent to those skilled in the art. Such variations are intended to be within the scope of this invention.

1. A method for trafficking cells comprising the steps of:
a) providing a surface having nanotubes immobilized thereon, said nanotubes selected from the group consisting of halloysite, silica and titanium oxide and having cell adhesion molecules immobilized on the outer surface thereof; and
b) flowing a fluid comprising a mixture of cells over the surface, wherein cells expressing ligands to cell adhesion molecules on the cell surface exhibit rolling and thereby flow at a different velocity than cells that do not express ligands to cell adhesion molecules on the cell surface allowing separation of the ligand expressing cells from the cells that do not express the ligands.

2. The method of claim 1, wherein the cell adhesion molecules are selected from the group consisting of selectins, cadherins, integrins and GP1b, and fragments thereof.

3. The method of claim 2, wherein the selectins are selected from the group consisting of P-selectin, L-selectin and E-selectin.

4. The method of claim 1, wherein the inner diameter of the flow device is in the range of 10-500 microns to promote margination of target cells towards the device surface.

5. The method of claim 1, wherein one of the cells in the mixture of cells is stem cell or cancer cell.

6. The method of claim 1, wherein step b) comprises subjecting the cells to a shear stress of 0.5 to 10 dynes/cm².

7. The method of claim 1, wherein the nanotubes are halloysite nanotubes and the majority of nanotubes have a length of 500 to 1200 nm and a diameter of 40 to 200 nm.

8. A device for cell trafficking comprising a fluidic flow chamber having nanotubes immobilized on the inner surface of the chamber, said nanotubes selected from the group consisting of halloysite, silica and titanium oxide and said nanotubes having cell adhesion molecules immobilized on the outer surface.

9. The device of claim 8, wherein a composition comprising positively charged molecules is coated on the inner surface of the chamber prior to immobilizing the nanotubes thereon.

10. The device of claim 8, wherein the positively charged molecules are poly L-lysine or titanium butoxide.

11. The device of claim 8, wherein the nanotubes are halloysite nanotubes and the length of majority of the nanotubes is from 500 nm to 1.2 μm and the diameter is 40 nm to 200 nm.

12. The device of claim 8, wherein the nanotubes project into the lumen of the fluidic flow chamber up to a height of 1.2 μm.

13. The device of claim 12, wherein the nanotubes project into the lumen of the fluidic flow chamber up to a height less than 900 nm.

14. The method of claim 8, wherein the cell rolling molecules are selected from the group consisting of selectins, cadherins, integrins and GP1b and fragments thereof.

15. The method of claim 14, wherein the selectins are selected from the group consisting of P-selectin, L-selectin and E-selectin.

16. The device of claim 8, wherein the nanotubes carry a delivery molecule in the lumen.

17. The device of claim 16, wherein the delivery molecule is a cationic molecule.

18. A method for making a device for cell trafficking comprising the steps of:
a) providing a fluid flow chamber;
b) providing nanotubes selected from the group consisting of halloysite, silica and titanium oxide, said nanotubes being substantially free of aggregates;
c) attaching cell adhesion molecules to the outer surface of the nanotubes;
d) attaching the nanotubes from c) to a flow surface of the fluid flow chamber.
